(19) United States
(12) Patent Application Publication
(10) Pub. No.: US 2011/0282389 A1
(43) Pub. Date: Nov. 17, 2011

(54) Bone fixation plate assembly

(75) Inventors: David Janice, Austin, TX (US); David William Strausser, Woodlands, TX (US)

(73) Assignee: Omni Surgical dba Spine 360

(21) Appl. No.: 12/800,487

(22) Filed: May 17, 2010

(51) Int. Cl. A61B 17/70; A61B 17/80

(52) U.S. Cl. 606/264; 606/291

(57) ABSTRACT

A bone fixation plate assembly includes an elongated plate having two or more sets of two adjacent screw holes extending between its top and bottom surfaces. A landing extends through the top surface of between the adjacent screw holes of a set. An interior sidewall of each one of the screw holes is spherically shaped. Each one of the screw holes has a minimum diameter at its bottom edge that is less than a minimum diameter at its top edge. An anti-back-out device is fixedly attached to the elongated plate on each one of the landings. Opposing end portions of each anti-back-out device has one or more screw engaging structures integral therewith overhanging an adjacent one of the screw holes at the top edge thereof. The one or more screw engaging structures is deflectable with respect to a mounting portion of a respective one of the anti-back-out devices.

### FIELD OF THE DISCLOSURE

The disclosures made herein relate generally to spinal fixation and, more particularly, to cervical plates used for immobilizing cervical vertebrae in the treatment of spinal disorders.

### BACKGROUND

It is known to provide an anterior cervical plate for attachment to the anterior of two or more cervical vertebrae for the purpose of immobilizing, stabilizing and/or aligning those vertebrae. The plates can be used for a variety of conditions including, for example, providing added stability, strength and/or rigidity to secure vertebrae together after fusion of adjacent vertebrae. Such a procedure is often performed for reasons such as, for example, correcting spinal deformities and/or correcting instability caused by trauma, tumors, advanced degenerative discs, infection or congenital or acquired deformities.

Cervical plates are generally elongated so as to span the distance between two, three, four or more vertebrae, as required in a given situation. The plates are generally curved transversely so as to fit the curvature of the vertebrae to which they are attached. Additionally, such cervical plates can also be concave longitudinally to match the curvature of the cervical spine. Cervical plates generally include screw holes in which screws are received. Screw holes are drilled into the adjacent vertebrae (i.e., bone), after which, the cervical plate is secured in place by bone screws which pass through the screw holes in the cervical plate and are fastened into the screw holes drilled in the vertebrae.

A common shortcoming with cervical plates is the backing out (i.e., excessive loosening) of the screws used to secure them in place. Anatomical stresses from body movements contribute greatly to the problem of such screw back-out, thereby jeopardizing the integrity of fixation. For example, slight shock or vibration of the vertebrae such as, for example, due to walking, climbing stairs or more vigorous activity by the patient following treatment increases the tendency for screw back-out. If the screws loosen, the bones are not properly secured and can move relative to each other. This can compromise the ability to achieve optimal bone fusion and bone alignment, or it may lead to loss of graft material, and damage or loss of bone. Moreover, as the fasteners work loose, the outward protrusion of the screw heads can be a source of discomfort and present the risk of trauma to adjacent and/or surrounding soft tissue.

### SUMMARY OF THE DISCLOSURE

Embodiments of the present invention relate to plates used in fixation of bones. In one specific implementation, a cervical plate is provided for immobilizing adjacent vertebrae bodies. Advantageously, embodiments of the present invention are configured in a manner for allowing bone screws to be selectively retracted from within mating screw holes of the cervical plate while simultaneously limiting the potential for unrestricted back-out of such screws due to vibrations, shocks and the like.

In one embodiment of the present invention, a plate assembly for immobilizing adjacent vertebrae bodies comprises an elongated plate sized to span between at least two vertebrae and an anti-back-out device. The elongated plate has two adjacent screw holes extending therethrough between a top surface and a bottom surface thereof. A landing extends through the top surface of the plate between the adjacent screw holes. An interior sidewall of each one of the screw holes is spherically shaped. Each one of the screw holes has a minimum diameter at a bottom edge thereof adjacent the bottom surface of the plate that is less than a minimum diameter at a top edge thereof adjacent the top surface of the plate. The anti-back-out device is fixedly attached to the elongated plate on the landing. Opposing end portions of the anti-back-out device has one or more screw engaging structures integral therewith overhanging the screw holes at the top edge thereof. The one or more screw engaging structures is deflectable with respect to a mounting portion of a respective one of the anti-back-out device.

In another embodiment of the present invention, an apparatus for immobilizing adjacent vertebrae bodies comprises a cervical plate, a plurality of anti-back-out devices, and a plurality of bone screws. The cervical plate includes two or more sets of two adjacent screw holes extending therethrough between a top surface and a bottom surface of the cervical plate. A landing extends through the top surface of the cervical plate between the adjacent screw holes of a set. An interior sidewall of each one of the screw holes is spherically shaped. Each one of the screw holes has a minimum diameter at a bottom edge thereof adjacent the bottom surface of the cervical plate that is less than a minimum diameter at a top edge thereof adjacent the top surface of the cervical plate. Each one of the anti-back-out devices is fixedly attached to the cervical plate on a respective one of the landings. Opposing end portions of the anti-back-out devices each has a resilient screw engaging structure integral therewith overhanging an adjacent one of the screw holes at the top edge thereof. Each one of the bone screws has a spherically shaped head portion and a shank portion attached to the head portion. The head portion of each one of the screws is captured within the spherically shaped interior sidewall of a respective one of the screw holes thereby allowing the head portion to pivot therein.

In another embodiment of the present invention, a component system for immobilizing adjacent vertebrae bodies comprises a cervical plate assembly and a plurality of bone screws. The cervical plate assembly includes a cervical plate and a plurality of anti-back-out devices. The cervical plate includes two or more sets of two adjacent screw holes extending therethrough between a top surface and a bottom surface thereof. A substantially flat landing extends through the top surface of the plate between the adjacent screw holes of a set. An interior sidewall of each one of the screw holes is spherically shaped. Each one of the screw holes has a minimum diameter at a bottom edge thereof adjacent the bottom surface of the cervical plate that is less than a minimum diameter at a top edge thereof adjacent the top surface of the cervical plate. Each one of the anti-back-out devices is fixedly attached to the cervical late on a respective one of the landings. Opposing end portions of the anti-back-out devices each has a resilient screw engaging structure integral therewith overhanging an adjacent one of the screw holes at a top edge thereof. Each one of the bone screws has a spherically shaped head portion and a shank portion attached to the head portion. The head portion of each one of the screws is sized for being captured, rotated and pivoted within the spherically shaped interior sidewall of any one of the screw holes.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is a top perspective view of a cervical plate system configured in accordance with an embodiment of the present invention.

FIG. 2 is a bottom perspective view of the cervical plate system shown in FIG. 1.

FIG. 3 is a cross sectional view taken along the line 3-3 in FIG. 1.

FIG. 4 is a perspective view of a bone screw of the cervical plate system shown in FIG. 1.

### DETAILED DESCRIPTION OF THE DRAWING FIGURES

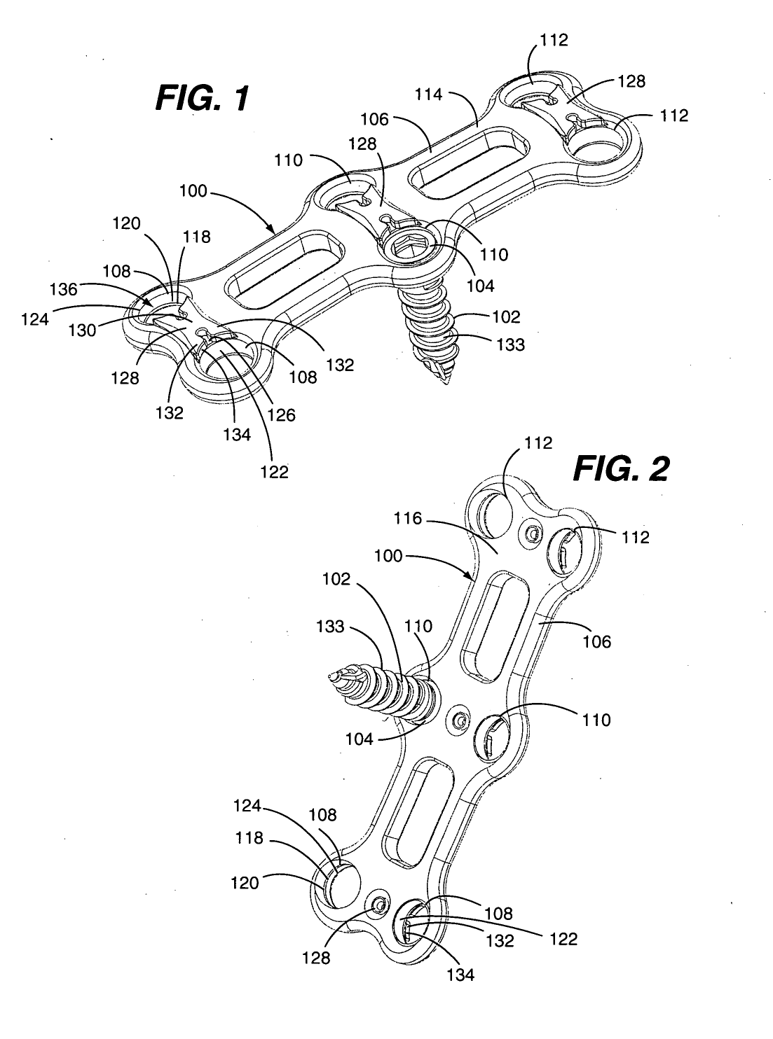

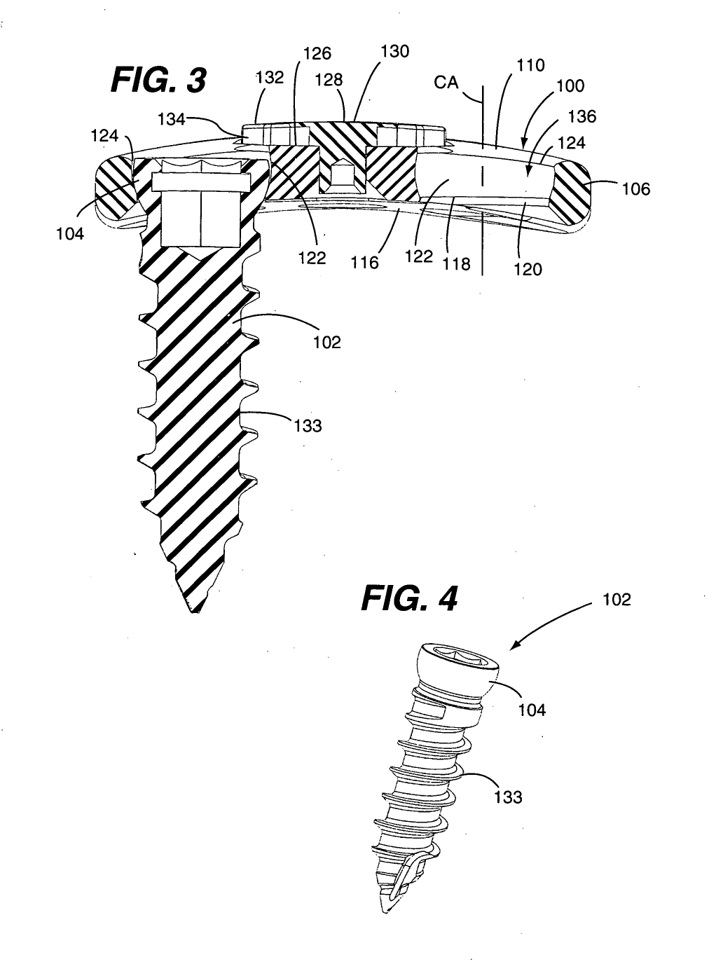

Referring now to FIGS. 1-3, a cervical plate assembly 100 and bone screw 102 configured in accordance with an embodiment of the present invention is shown. The cervical plate assembly 100 is an example of an apparatus for immobilizing adjacent vertebrae bodies. Jointly, the cervical plate assembly 100 and a plurality of the bone screws 102 are a cervical plate system configured in accordance with an embodiment of the present invention. Accordingly, the cervical plate is an embodiment of a bone fixation plate configured in accordance with an embodiment of the present invention.

Advantageously, the cervical plate assembly 100 is configured in a manner for allowing a bone screw 102 thereof to be selectively retracted from within mating screw holes of the cervical plate while simultaneously limiting the potential for unrestricted back-out of such screws due to vibrations, shocks and the like. Limiting the potential for unrestricted back-out of the bone screw 102 and other bone screws of the cervical plate assembly 100 (not shown) enhances the ability to achieve optimal bone fusion and bone alignment, retention of graft material, and reduced potential for damage or loss of bone at the placement sight of the cervical plate assembly. Furthermore, limiting the potential for such unrestricted back-out of the bone screw 102 reduces the potential for a head portion 104 of the bone screw 102 to become a source of discomfort and present the risk of trauma to adjacent and/or surrounding soft tissue.

A cervical plate 106 of the cervical plate assembly 100 includes a plurality of sets of screw holes 108, 110, 112. Each one of the sets of screw holes 108, 110, 112 includes two adjacent pair of screw holes (i.e., two side-by-side screw holes) extending through the cervical plate 106 between a top surface 114 and a bottom surface 116 of the cervical plate 106. Preferably, but not necessarily, the cervical plate 106 can be generally curved transversely so as to fit the curvature of the cervical spine vertebrae and/or can be concave longitudinally to match the curvature of the cervical spine.

It is disclosed herein that a cervical plate or other type of immobilizing apparatus configured in accordance with the present invention is not unnecessarily limited to a particular number of sets of adjacent screw holes. Accordingly, a cervical plate or other type of immobilizing apparatus configured in accordance with the present invention can have two or more sets of adjacent screw holes (e.g., two or more pairs of adjacent screw holes). It is also possible for an immobilizing apparatus configured in accordance with the present invention to have only one set of adjacent screw holes (e.g., one pair of screw holes).

Each one of the screw holes 108, 110, 112 is bounded at its bottom edge 118 adjacent the bottom surface 116 of the cervical plate 106 by a constant diameter shoulder 120. An interior sidewall 122 of each one of the screw holes 108, 110, 112 is spherically shaped. Each one of the screw holes 108, 110, 112 has a minimum diameter at a bottom edge 118 of the interior sidewall 122 that is less than a minimum diameter at a top edge 124 of the interior sidewall 122. Stated differently, but with the same resulting construction, each one of the screw holes 108, 110, 112 has a minimum diameter at its bottom edge 118 adjacent the bottom surface 116 of the cervical plate 106 that is less than a minimum diameter at its top edge 124 adjacent the top surface 114 of the cervical plate 106. Such diametrical dimensions are referenced with respect to a centerline axis CA of a respective one of the screw holes 108, 110, 112.

A landing 126 extends through the top surface 114 of the cervical plate 106 between each set of screw holes 108, 110, 112. Each landing 126 can intersect the corresponding set of screw holes 108, 110, 112, thereby partially defining the top edge 124 thereof. Preferably, but not necessarily, each one of the landings 126 is substantially flat and the centerline axis CA of each set of screw holes 108, 110, 112 extends substantially perpendicular to a respective one of the landings 126.

An anti-back-out device 128 is fixedly attached to the cervical plate 106 on each one of the landings 126. Each anti-back-out device 128 extends between an adjacent set of the screw holes 108, 110, 112. In one embodiment, each anti-back-out device 128 is a plate that can be substantially flat on one of both of its major surfaces.

Each anti-back-out device 128 has a mounting portion 130 and has a pair of screw engaging structures 132 at opposing ends of the mounting portion 130. The screw engaging structures 132 are resiliently integral with the mounting portion 130 of the respective anti-back-out device 128 (i.e., are resilient screw engaging structures). Each pair of screw engaging structure 132 overhangs the adjacent one of the screw holes 108, 110, 112 at its top edge 124. The screw engaging structures 132 are shaped such that application of force on an end face 134 of a particular one of the screw engaging structures 132 causes the particular one of the screw engaging structures 132 to deflect with respect to the respective mounting portion 130. This deflection reduces the amount that the particular one of the screw engaging structure 132 overhangs the adjacent one of the screw holes 108, 110, 112.

Each one of the screw holes 108, 110, 112 can have the head portion 104 of the bone screw 102) seated therein with a shank portion 133 extending below the bottom surface 116 of the cervical plate 106. The head portion 104 of the bone screw 102 is a spherically-shaped and has dimensions that allow the head portion 104 to be matingly engaged within a head-receiving space 136 defined by the spherically shaped interior sidewall 122 of any one of the screw holes 108, 110, 112. Such mating engagement refers to the spherically-shaped head portion 104 being sized to pivot within the head-receiving space 136 without excessive play or bind (i.e., as would a known ball-socket type joint). Furthermore, a maximum diameter of the spherically-shaped head portion 104 is slightly larger than the diameter of the top edge 124 of the screw holes 108, 110, 112 such that the spherically-shaped head portion 104 must be forcibly urged past the top edge 124 of a particular one of the screw holes 108, 110, 112 when being seated within the head-receiving space 136. Though this construction, the head portion 104 of the bone screw can be captured within the head-receiving space 136 defined by the spherically shaped interior sidewall 122 thereby allowing the head portion 104 to pivot within the head-receiving space 136.

In one example of use, a cervical plate in accordance with an embodiment of the present invention is positioned across a space between two adjacent vertebrae bodies. In this position, a first set of screw holes of the cervical plate are aligned with an anterior surface of a first one of the adjacent vertebrae bodies and a second set of screw holes of the cervical plate are aligned with an anterior surface of a second one of the adjacent vertebrae bodies. Pilot holes are then drilled through each one of the screw holes into the underlying one of the vertebrae bodies. A bone screw is then screwed into the corresponding pilot hole through each one of the screw holes. Sufficient screwing of each bone screw into the vertebrae causes the head portion of each screw to be urged past the screw engaging structures of the adjacent anti-back-out device (i.e., via the above-mentioned deflection of the screw engaging structures), then to be urged through the top edge of a particular one of the screw holes, and finally to be seated within the head-receiving space of the particular one of the screw holes against the spherically-shaped interior sidewall of the screw hole. Engagement of the head portion of the screws with the spherically-shaped interior sidewall of the screw holes causes the first and second vertebrae to be held in effectively fixed position with respect to the cervical plate, thereby immobilizing adjacent vertebrae bodies. The top edge of the screw holes and the overhanging portion of the screw engaging structures both serve as independent means for limit unrestricted back-out of the bone screws once tightened into the vertebrae body or other engaged bony structure. For example, in the event that a screw backs out past the top edge of the screw holes, the overhanging portion of the respective screw engaging structures will provide additional means for limiting the potential for further unrestricted back-out of the bone screw.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A bone fixation plate assembly for immobilizing adjacent vertebrae bodies, comprising:
an elongated plate sized to span between at least two vertebrae, wherein said plate has two adjacent screw holes extending therethrough between a top surface and a bottom surface thereof, wherein a landing extends through the top surface of said plate between said adjacent screw holes, wherein an interior sidewall of each one of said screw holes is spherically shaped, and wherein each one of said screw holes has a minimum diameter at a bottom edge thereof adjacent the bottom surface of said plate that is less than a minimum diameter at a top edge thereof adjacent the top surface of said plate; and
an anti-back-out device fixedly attached to the elongated plate on the landing, wherein opposing end portions of the anti-back-out device has at least one screw engaging structure integral therewith overhanging an adjacent one of said screw holes at the top edge thereof and wherein said at least one screw engaging structure is deflectable with respect to a mounting portion of a respective one of said anti-back-out devices.

2. The bone fixation plate assembly of claim 1 wherein:
the landing has a substantially flat surface; and
a centerline axis of each one of said screw holes extends substantially perpendicular to the substantially flat surface of the landing.

3. The bone fixation plate assembly of claim 2 wherein each one of said screw holes is bounded at the bottom edge thereof by a constant diameter shoulder.

4. The bone fixation plate assembly of claim 1 wherein:
the anti-back-out device is a substantially flat plate;
said at least one screw engaging structure includes two opposing screw biasing arms each overhanging the adjacent one of said screw holes at the top edge thereof; and
each one of said screw biasing arms is resiliently attached to the mounting portion of the anti-back-out device.

5. The bone fixation plate assembly of claim 1 wherein each one of said screw holes is bounded at the bottom edge thereof by a constant diameter shoulder.

6. The bone fixation plate assembly of claim 1 wherein the landing intersects the screw holes thereby partially defining the top edge thereof.

7. The bone fixation plate assembly of claim 6 wherein:
each one of said screw holes is bounded at the bottom edge thereof by a constant diameter shoulder;
said at least one screw engaging structure includes two opposing screw biasing arms each overhanging the adjacent one of said screw holes at the top edge thereof;
each one of said screw biasing arms is resiliently attached to the mounting portion of the respective one of said anti-back-out devices;
the landing has a substantially flat surface; and
a centerline axis of each one of said screw holes extends substantially perpendicular to the substantially flat surface of the landing.

8. An apparatus for immobilizing adjacent vertebrae bodies, comprising:
a cervical plate including at least two sets of two adjacent screw holes extending therethrough between a top surface and a bottom surface thereof, wherein a landing extends through the top surface of the cervical plate between said adjacent screw holes of a set, wherein an interior sidewall of each one of said screw holes is spherically shaped, and wherein each one of said screw holes has a minimum diameter at a bottom edge thereof adjacent the bottom surface of the cervical plate that is less than a minimum diameter at a top edge thereof adjacent the top surface of the cervical plate;
a plurality of anti-back-out devices each fixedly attached to the cervical plate on a respective one of said landings, wherein opposing end portions of said anti-back-out devices each has at least one resilient screw engaging structure integral therewith overhanging an adjacent one of said screw holes at the top edge thereof; and
a plurality of bone screws each having a spherically shaped head portion and a shank portion attached to said head portion, wherein said head portion of each one of said screws is captured within said spherically-shaped interior sidewall of a respective one of said screw holes thereby allowing said head portion to pivot therein.

9. The apparatus of claim 8 wherein:
each one of the landings has a substantially flat surface; and
a centerline axis of each one of said screw holes extends substantially perpendicular to the substantially flat surface of all of said landings.

10. The apparatus of claim 9 wherein each one of said screw holes is bounded at the bottom edge thereof by a constant diameter shoulder.

11. The apparatus of claim 8 wherein:
each one of said anti-back-out devices is a substantially flat plate;
said at least one resilient screw engaging structure includes two opposing resilient screw biasing arms each overhanging the adjacent one of said screw holes at the top edge thereof; and
each one of said resilient biasing arms is resiliently attached to a mounting portion of the respective one of said anti-back-out devices.

12. The apparatus of claim 8 wherein each one of said screw holes is bounded at the bottom edge thereof by a constant diameter shoulder.

13. The apparatus of claim 8 wherein each landing intersects a respective set of said screw holes thereby partially defining the top edge thereof.

14. The apparatus of claim 13 wherein:
each one of said screw holes is bounded at the bottom edge thereof by a constant diameter shoulder;
the resilient screw engaging structure includes two opposing resilient screw biasing arms each overhanging the adjacent one of said screw holes at the top edge thereof;
each one of said resilient biasing arms is resiliently attached to a mounting portion of the respective one of said anti-back-out devices;
each one of the landings has a substantially flat surface; and
a centerline axis of each one of said screw holes extends substantially perpendicular to the substantially flat surface of all of said landings.

15. A component system for immobilizing adjacent vertebrae bodies, comprising:
a cervical plate assembly including a cervical plate and a plurality of anti-back-out devices, wherein the cervical plate includes at least two sets of two adjacent screw holes extending therethrough between a top surface and a bottom surface thereof, wherein a substantially flat landing extends through the top surface of said plate between said adjacent screw holes of a set, wherein an interior sidewall of each one of said screw holes is spherically shaped, wherein each one of said screw holes has a minimum diameter at a bottom edge thereof adjacent the bottom surface of the cervical plate that is less than a minimum diameter at a top edge thereof adjacent the top surface of the cervical plate, wherein each one of said anti-back-out devices is fixedly attached to the cervical late on a respective one of said landings, and wherein opposing end portions of said anti-back-out devices each has a resilient screw engaging structure integral therewith overhanging an adjacent one of said screw holes at a top edge thereof; and
a plurality of bone screws each having a spherically shaped head portion and a shank portion attached to said head portion, wherein said head portion of each one of said screws is sized for being captured, rotated and pivoted within said spherically shaped interior sidewall of any one of said screw holes.

16. The component system of claim 15 wherein a centerline axis of each one of said screw holes extends substantially perpendicular to the substantially flat surface of all of said landings.

17. The component system of claim 16 wherein each one of said screw holes is bounded at the bottom edge thereof by a constant diameter shoulder.

18. The component system of claim 15 wherein:
each one of said anti-back-out devices is a substantially flat plate;
the resilient screw engaging structure includes two opposing resilient screw biasing arms each overhanging the adjacent one of said screw holes at the top edge thereof
each one of said resilient biasing arms is resiliently attached to a mounting portion of the respective one of said anti-back-out devices.

19. The component system of claim 15 wherein each one of said screw holes is bounded at the bottom edge thereof by a constant diameter shoulder.

20. The component system of claim 15 wherein each landing intersects a respective set of said screw holes thereby partially defining the top edge thereof.
